(19) United States
(12) Patent Application Publication
(10) Pub. No.: US 2007/0072947 A1
(43) Pub. Date: Mar. 29, 2007

(54) Use of deoxypeganine for treating schizophrenic psychoses

(75) Inventors: Joachim Moormann, Werne (DE); Klaus Opitz, Munster (DE)

Correspondence Address: D. PETER HOCHBERG CO. L.P.A., 1940 EAST 6TH STREET, CLEVELAND, OH, 44114, US

(73) Assignee: HF ARNEIMITTELFORSCHUNG GMBH, WERNE (DE)

(21) Appl. No.: 10/580,443

(22) PCT Filed: Nov. 8, 2004
(86) PCT No.: PCT/EP04/12605

§ 371 Date: May 23, 2006

(30) Foreign Application Priority Data
Nov. 24, 2003 (DE) 103 54 893.9

(51) Int. Cl. A61K 31/137

(52) U.S. Cl. 514/649

(57) ABSTRACT

Deoxypeganine in the form of a free base or in the form of an acid addition salt, or a derivative of deoxypeganine as long as the derivative is simultaneously an inhibitor of acetylcholinesterase and of monoamine oxidase. The deoxypeganine can be used for producing a medicament for treating a schizophrenic psychosis.

### CROSS-REFERENCE TO RELATED APPLICATIONS

This application is a National Stage application of International Application No. PCT/EP2004/012605, filed on Nov. 8, 2004, which claims priority of German application number 103 54 893.9, filed on Nov. 24, 2003.

### BACKGROUND OF THE INVENTION

1. Field of the Invention

The invention relates to deoxypeganine. More particularly, the present invention relates to the use of deoxypeganine as a free base or as an acid addition salt, or a derivative of deoxypeganine, in a medicament for treating schizophrenic psychoses.

2. Description of the Prior Art

Schizophrenia is a far-reaching endogenous psychiatric illness (psychosis) which is accompanied by changes in thinking, perception and behaviour of those affected.

In the case of schizophrenic psychosis there may be changes in practically every psychic function. A large number of complaints occur which need not be of the same intensity in all schizophrenia patients. Basically, with schizophrenias a distinction is made between fundamental symptoms and accessory symptoms.

Among the fundamental symptoms, which are disorders caused directly by the schizophrenic psychosis, are the blocking of thought processes, disorders of emotional life (affect) and drive, loss of reality (autism) and the so-called “ego disorder” which is understood to mean the split experience of ones own personality.

Among the accessory symptoms, i.e. complaints which may be developed by the schizophrenic patients in connection with the fundamental symptoms, are delusions, hallucinations, mannerism and megalomania.

Psychotic patients lose their ability to intellectually and emotionally communicate with other people and to realistically assess ongoing events as to their content and significance. What is essential is that schizophrenics do not think in a logical way connecting causes and effects which correspond to events in the real world. For example, schizophrenic patients may have bizarre delusions which lack any relation to reality. Schizophrenics also experience hallucinations, which usually are of acoustic nature.

Apart from the mentioned blocking of thought processes, schizophrenia is also accompanied by serious emotional impairments in a large number of those affected and they frequently suffer from lack of contact and are afraid of dealing with other people.

The above-mentioned symptoms of schizophrenia are to be distinguished from emotional disorders that do not only occur in connection with schizophrenia. Among these “non-schizophrenic” symptoms are anxiety, tension, agitation, feelings of guilt, depression, disorientation and psychosomatic symptoms.

The types of complaints in schizophrenia and in other emotional disorders are very similar and can frequently not be distinguished. For this reason a catalogue with specific guidelines for the diagnosis of schizophrenia has been developed based on the lack or occurrence of concrete behaviour patterns which can be easily observed. Thus, according to the guidelines of the Deutsche Gesellschaft für Psychiatrie, Psychotherapie und Nervenheilkunde (DGPPN) at least one unambiguous symptom from the group of symptoms consisting of

1. thought hearing; thought insertion or thought withdrawal, spreading of one's thoughts to others;

2. delusion of being controlled, delusion of being influenced, feeling of having done something—relating to body movements, thoughts, activities or sensations; interpretation delusion;

3. commenting or dialogic voices; and

4. continuing, culturally inadequate and entirely unrealistic delusion is necessary for diagnosing a schizophrenia, or there must be at least two symptoms from the group consisting of;

5. continuing hallucination of all sense modalities;

6. thought blocking or insertions in the flow of thoughts;

7. catatonic symptoms such as agitation, stereotypes of posture; negativism or stupor; and

8. “negative” symptoms such as conspicuous apathy, impoverishment of speech, flattened or inadequate affects.

It has to be taken into account in this connection that the causes of schizophrenic symptoms must also be distinguished from other possibilities of symptom development such as, for example, drug and medicament abuse, brain tumours and other neurological diseases.

It is estimated that 1% of the world population suffers from classic schizophrenia, that is, from a form of this psychosis where the symptoms are so massive and unambiguous that there can be no doubt as to the diagnosis.

The exact causes of a schizophrenic disorder are still unknown, but the chemical messenger substances which transmit the nerve signals (neurotransmitters) play an important role. In the past it was presumed that schizophrenia was a consequence of the overproduction of the neurotransmitter dopamine. Later studies, however, indicated that part of the signal transduction paths of the dopamine is overactive. It was proved, for example, that the neuroleptics used for treating schizophrenia cancel the effect of dopamine in the brain by binding to postsynaptic dopamine receptors.

In the large majority of corresponding studies a decreased or unaltered monoaminoxidase activity has been observed in schizophrenia patients as compared to non-schizophrenic probands, which is in correspondence with the hypothesis of an overactive dopamine signal transduction path. By contrast to the majority of studies, Lewine and Meltzer were able to prove a significant positive correlation between the negative symptoms and the activity of the monoaminoxidase from the thrombocytes of male, unmedicated schizophrenics (Lewine, R. J. and Meltzer; H. Y., Psychiatry Res. 12, 99-109 (1984)). Schildkraut and his collaborators also found an increased monoaminoxidase activity in the thrombocytes of patients suffering from schizophrenia-related depression (Schildkraut et al., Schizophr. Bull. 6, 220-225(1980); Schildkraut et al., Am J Psychiatry 135, 110-112(1978)).

Apart from the above, there have more recently been indications from epidemiological analyses and behavioural studies which lead one to assume that neuronal nicotinic receptors (nAChRs) also play a role in the pathogenesis of neurological disorders, including schizophrenia. There have, for example, been reports on a decrease in nicotinic acetylcholine receptors in schizophrenics, and especially the alpha 7 subtype of the nicotinic acetylcholine receptors is regarded as being relevant for a schizophrenic disorder. These observations lead to interest in allosteric modulators of nicotinic acetylcholine receptors, such as, for example, galanthamine, for the treatment of neurological disorders which can be connected with a change in the function or expression of nicotinic acetylcholine receptors (Deutsch, S. I. et al., Life Sciences 73, 2333-2361 (2003)).

Apart from the psychotherapeutic care, the pharmacotherapy with antipsychotics, predominantly with neuroleptics, forms the basis of the treatment of schizophrenic psychoses. By administering psychopharmacological agents it is possible to alleviate the symptoms of schizophrenia. Neuroleptics can decrease tension and enable the patient to deal, beyond his delusion, with other people so that the prognosis is favourable for more than 50% of those suffering from schizophrenia. These patients are again able to integrate themselves in their social environment, and also to work again. Psychopharmacological agents are, however, not able to heal schizophrenia.

Existing pharmacotherapies in addition have the disadvantage that considerable side effects occur. Thus, the neuroleptics had a long list of severe side effects such as motor disturbance, involuntary muscular twitching, dullness of feeling, tiredness, lack of drive, and weight gain, most of which, with the exception of involuntary muscular twitching, disappear after discontinuing the medicament, it is true, but the fact that these side effects occur also reveals that there is still a need for better pharmacotherapeutic agents that have fewer side effects or whose side effects are not as severe, or by means of which the symptoms of a schizophrenic psychosis cannot only be suppressed but the illness even be healed.

### SUMMARY OF THE INVENTION

The object of the present invention was therefore to provide active substances for developing improved psychopharmacological agents for the treatment of schizophrenic psychoses.

According to the invention this object is achieved by using deoxypeganine for treating schizophrenia or using deoxypeganine for the manufacture of medicaments for treating schizophrenia.

Deoxypeganine (1,2,3,9-tetrahydropyrrolo[2,1-b]quinazoline) is an alkaloid of molecular formula C₁₁H₁₂N₂ which occurs in plants of the Zygophyllaceae family. Deoxypeganine is preferably obtained by isolation from Syrian rue (Peganum harmala) or by chemical synthesis. It is known to the pharmaceutical art from the literature and, in particular, from patent specifications.

DE-A 199 06 978, respectively WO 00/48582, describes medicaments based on deoxypeganine for the therapy of drug addiction and drug dependence.

DE-A 199 06 979, respectively WO 00/48445, describes medicaments based on deoxypeganine for the therapy of nicotine dependence.

DE-A 199 06 975, respectively WO 00/48599, describes the use of deoxypeganine for the therapy of Alzheimer's dementia.

DE-A 101 63 667, respectively WO 03/053445 discloses the use of deoxypeganine for treating clinical depression.

Based on its pharmacological properties, deoxypeganine is included in the group of reversibly acting cholinesterase inhibitors. The fact that deoxypeganine does not only inhibit acetylcholinesterase but also monoamine oxidases, is in general terms known from the above-indicated publications. The monoamine oxidase-inhibiting action of deoxypeganine is in all of these documents described as a merely complementary action which is intended to reinforce the acetylcholinesterase-inhibiting action of deoxypeganine, the latter inhibition being regarded as most important.

Because of its double mechanism of action, deoxypeganine is said to be intended preferably for use in the treatment of a schizophrenic psychosis, or for use in the manufacture of a medicament for treating a schizophrenic psychosis that is connected with increased monoaminoxidase activity and/or decreased functionality (decreased activity or decreased expression) of nicotinic acetylcholine receptors, especially of the alpha 7 subtype.

Administration of deoxypeganine may be peroral or parenteral. For oral administration known administration forms can be used such as tablets, coated tablets, capsules, lozenges. Also suitable are liquid or semiliquid dosage forms, for example as drinking solutions, in which case the agent is present in the form of a solution or suspension. Solvents or suspending agents that can be used are water, aqueous media or pharmacologically acceptable oils (vegetable or mineral oils).

The deoxypeganine-containing medicaments are preferably formulated as depot drugs which are able to deliver this agent to the body in a controlled manner and over an extended period.

Moreover, deoxypeganine may according to the invention also be administered rectally (e.g. by introducing suppositories), inhalationally (by breathing in aerosols with defined concentration and size distribution of the particles), transdermally (by active agent-containing patches, liniment solutions, gels etc.), transmucosally (in the sense of absorption through the oral and nasal mucous membranes, with the active agent being released in the oral cavity by dissolution in saliva or being brought into the nose by spray solutions and the like), by implanted vessels (which release the active agent passive-osmotically or in a controlled manner by minipumps or the like), by intravenous, intramuscular or subcutaneous injection and intracerebroventricularly.

In connection with the parenteral administration, it is particularly advantageous to use transdermal or transmucosal dosage forms for the deoxypeganine administration according to the invention, in particular adhesive transdermal therapeutic systems (active agent plasters) as described specifically for deoxypeganine in DE-A 199 06 977. These enable the delivery of the agent in a controlled manner over a prolonged period via the skin to the patient being treated.

### DETAILED DESCRIPTION OF THE INVENTION

According to the invention, deoxypeganine can be used both in the form of its free base and as acid addition salt for treatment; preferred salts are deoxypeganine hydrochloride and deoxypeganine hydrobromide. In addition, it is also possible to use salts of other pharmacologically acceptable acids, e.g. citrate, tartrate or acetate.

In place of deoxypeganine, its derivatives described in the literature are also to be understood in a similar way as long as they are simultaneously inhibitors of acetylcholinesterase and of monoamine oxidases. These include the 7-bromodeoxypeganine described in Synthetic Communs. 25(4), 569-572 (1995), as well as the 7-halo-6-hydroxy-5-methoxydeoxypeganines which are described in Drug Des. Disc. 14, 1-14 (1996) and have the general formula 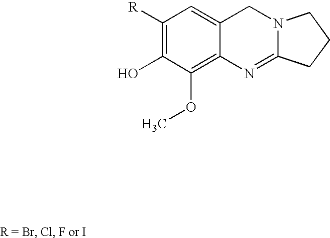
7-Bromo-6-hydroxy-5-methoxydeoxypeganine
7-Chloro-6-hydroxy-5-methoxydeoxypeganine
7-Fluoro-6-hydroxy-5-methoxydeoxypeganine
7-Iodo-6-hydroxy-5-methoxydeoxypeganine

The deoxypeganine derivatives described in Ind. J. Chem. 24B, 789-790 (1985) can also furthermore be used, namely 1,2,3,9-tetrahydro-6,7-methylenedioxypyrrolo[2,1-b]chinazoline and 2,3-dihydro-6,7-dimethoxypyrrolo[2,1-b]quinazoline-9(1H)-on.

The pharmaceutical forms which can be used according to the present invention for administering deoxypeganine may comprise one or more of the following additives:

antioxidants, synergists, stabilizers;

preservatives;

taste corrigents;

colourants;

solvents, solubilizers;

surfactants (emulsifiers, solubilizers, wetting agents, antifoams);

agents affecting the viscosity and consistency, gel formers;

absorption promoters;

adsorbents, humectants, lubricants;

agents affecting disintegration and dissolution, fillers (extenders), peptizers;

release-delaying agents.

This list is not definitive; the suitable physiologically acceptable substances are known to the skilled person.

Deoxypeganine is preferably administered in a pharmaceutical preparation which contains the agent in proportions of from 0.1 to 90% by weight, particularly preferably in proportions of from 2 to 20% by weight, in each case calculated as free deoxypeganine. The deoxypeganine-containing pharmaceutical preparations used according to the invention may additionally contain additives, such as inactive ingredients or adjuvants, excipients or vehicles, and/or stabilizers, in the amounts known to the skilled person.

The dose administered each day is preferably in the range from 0.1 to 100 mg, in particular from 10 to 50 mg. It should be adjusted appropriately depending on the individual requirements.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. Use of deoxypeganine, in the form of a free base or in the form of an acid addition salt, or of a derivative of deoxypeganine as long as said derivative is simultaneously an inhibitor of acetylcholinesterase and of monoamine oxidase, for producing a medicament for treating a schizophrenic psychosis which is connected with at least one of increased monoamine oxidase activity and decreased functionality of nicotinic acetylcholine receptors.

2. The use according to claim 1, wherein the medicament contains the active substance deoxypeganine in proportions of 0.1 to 90%-wt calculated as free deoxypeganine.

3. The use according to claim 1, wherein said medicament has a depot effect.

4. The use according to claim 1, wherein said medicament can be administered orally.

5. The use according to claim 1, wherein said medicament can be administered parenterally.

6. The use according to claim 5, wherein said medicament can be administered transdermally.

7. Use of deoxypeganine, in the form of a free base or in the form of an acid addition salt, or of a derivative of deoxypeganine as long as said derivative is simultaneously an inhibitor of acetylcholinesterase and of monoamine oxidase, for treating a schizophrenic psychosis which is connected with at least one of increased monoamine oxidase activity and decreased functionality of nicotinic acetylcholine receptors.

8. The use according to claim 7, wherein the administered daily dose is in the range 0.1 to 100 mg.

9. The use according to claim 7, wherein deoxypeganine is administered in a pharmaceutical preparation containing the active substance in proportions of 0.1 to 90%-wt, calculated as free deoxypeganine.

10. The use according to claim 9, wherein deoxypeganine is administered in a pharmaceutical preparation having a depot effect.

11. The use according to claim 9, wherein deoxypeganine is administered orally.

12. The use according to claim 9, wherein deoxypeganine is administered parenterally.

13. The use according to claim 12, wherein deoxypeganine is administered transdermally.

14. The use according to claim 7, wherein said nicotinic acetylcholine receptors are nicotinic acetylcholine receptors of the alpha 7 subtype.

15. The use according to claim 7, wherein said derivative of deoxypeganine, as long as it is simultaneously an inhibitor of acetylcholinesterase and of monoamine oxidase, is selected from the group consisting of 7-bromodeoxypeganine, 7-bromo-6-hydroxy-5-methoxydeoxypeganine, 7-chloro-6-hydroxy-5-methoxydeoxypeganine, 7-fluoro-6-hydroxy-5-methoxydeoxypeganine, 7-iodo-6-hydroxy-5-methoxydeoxypeganine, 1,2,3,9-tetrahydro-6,7-methylenedioxypyrrolo[2,1-b]chinazoline and 2,3-dihydro-6,7-dimethoxypyrrolo[2,1-b]quinazoline-9(1H)-on.

16. The use according to claim 1, wherein the decreased functionality of nicotinic acetylcholine receptors is decreased activity or decreased expression.

17. The use according to claim 2, wherein the medicament contains the active substance deoxypeganine in proportions of 2 to 20%-wt, calculated as free deoxypeganine

18. The use according to claim 7, wherein the decreased functionality of nicotinic acetylcholine receptors is decreased activity or decreased expression.

19. The use according to claim 8, wherein the administered daily dose is in the range 10 to 50 mg.

20. The use according to claim 9, wherein deoxypeganine is administered in a pharmaceutical preparation containing the active substance in proportions of 2 to 20%-wt, calculated as free deoxypeganine.

21. The use according to claim 1, wherein said nicotinic acetylcholine receptors are nicotinic acetylcholine receptors of the alpha 7 subtype.

22. The use according to claim 1, wherein said derivative of deoxypeganine, as long as it is simultaneously an inhibitor of acetylcholinesterase and of monoamine oxidase, is selected from the group consisting of 7-bromodeoxypeganine, 7-bromo-6-hydroxy-5-methoxydeoxypeganine, 7-chloro-6-hydroxy-5-methoxydeoxypeganine, 7-fluoro-6-hydroxy-5-methoxydeoxypeganine, 7-iodo-6-hydroxy-5-methoxydeoxypeganine, 1,2,3,9-tetrahydro-6,7-methylenedioxypyrrolo[2,1-b]chinazoline and 2,3-dihydro-6,7-dimethoxypyrrolo[2,1-b]quinazoline-9(1H)-on.

23. A method for treating schizophrenic psychosis comprising the steps of:
preparing a medicament comprising an active substance selected from the group consisting of deoxypeganine and a derivative of deoxypeganine, wherein said deoxypeganine is in the form of a free base or an acid addition salt and said derivative of deoxypeganine is simultaneously an acetylcholinesterate inhibitor and a monoamine oxidase inhibitor, and wherein said active substance is provided in a proportion between 0.1 to 90%-wt calculated as free deoxypeganine;
administering said medicament in a manner selected from the group consisting of orally, parenterally, rectally, inhalationally, transmucosally and transdermally; and
administering said medicament in a daily dose in the range of 0.1 to 100 mg.

24. The method according to claim 23, wherein said acid addition salt is selected from the group consisting of deoxypeganine hydrochloride and deoxypeganine hydrobromide.
